(19) United States
(12) Plant Patent Application Publication
(10) Pub. No.: US 2026/0053078 P1
(43) Pub. Date: Feb. 19, 2026

(54) Slamka garlic: a hardy, disease-resistant garlic variety

(50) ;

(71) Applicant: Katrina Anne Leiber, Franksville, WI (US)

(72) Inventor: Katrina Anne Leiber, Franksville, WI (US)

(21) Appl. No.: 18/831,654

(22) Filed: Jul. 7, 2025

Related U.S. Application Data
(60) Provisional application No. 63/732,456, filed on Aug. 19, 2024.

(51) Int. Cl. A01H 6/04; A01H 5/00

(52) U.S. Cl. CPC A01H 6/04; A01H 5/00
USPC PLT/258

(57) ABSTRACT

A new and distinct variety of garlic plant, Allium sativum L., named “Slamka Garlic,” is characterized by its exceptional disease and pest resistance, high yields, minimal cultivation requirements, and robust hardiness. Originating from a strain cultivated for more than a century in Southeastern Wisconsin, this variety is particularly well-suited to temperate climates and has shown consistent resistance to common garlic diseases over an eight-year observation period. These traits make it an ideal choice for growers in temperate regions, particularly Southeastern Wisconsin. “Slamka Garlic” has not been tested in other regions and its ability to thrive outside of Southeastern Wisconsin is unknown.

### BACKGROUND

Garlic cultivation has a rich history, with evidence of its use and cultivation spanning thousands of years across various cultures. Despite its long history, garlic crops are vulnerable to numerous diseases and pests, which can significantly impact yields. To mitigate these challenges, garlic farmers may rely on pesticides and costly disease management techniques.

By addressing these challenges, Slamka Garlic offers a sustainable solution for growers seeking high-yield, low-maintenance garlic with natural disease resistance. The present invention introduces “Slamka Garlic,” a new garlic variety that does not require pesticides. It offers robust disease and pest resistance, minimal cultivation requirements, high yields, and exceptional overall resilience.

### SUMMARY OF INVENTION

“Slamka Garlic” is a distinct variety of Allium sativum L., discovered and cultivated exclusively in Southeastern Wisconsin without pesticides or other chemical treatments. This variety has been selectively propagated for over a century, resulting in a strain that is exceptionally hardy and well-adapted to the region's climate and soil conditions. It performs optimally in hilled rows topped with weed barrier and typically does not require supplemental watering except during germination and extreme droughts.

Slamka Garlic is characterized by its overall resilience, disease resistance, pest resistance, high yields, and minimal cultivation needs. No instances of disease have been observed in Slamka Garlic over an eight-year cultivation period in Southeastern Wisconsin, demonstrating its suitability for growers in this region and similar climates where conditions are comparable.

### BRIEF DESCRIPTION OF THE DRAWINGS

The attached photographs are of Slamka Garlic grown during the 2023-2024 growing season in Southeastern Wisconsin. The photographs show the overall appearance of the new variety Slamka Garlic. The colors in the photos are as accurate as reasonably possible but may differ slightly from the true color of the plant.

FIG. 1: Garlic plant pulled 9.5 months after planting, displayed next to a yardstick.

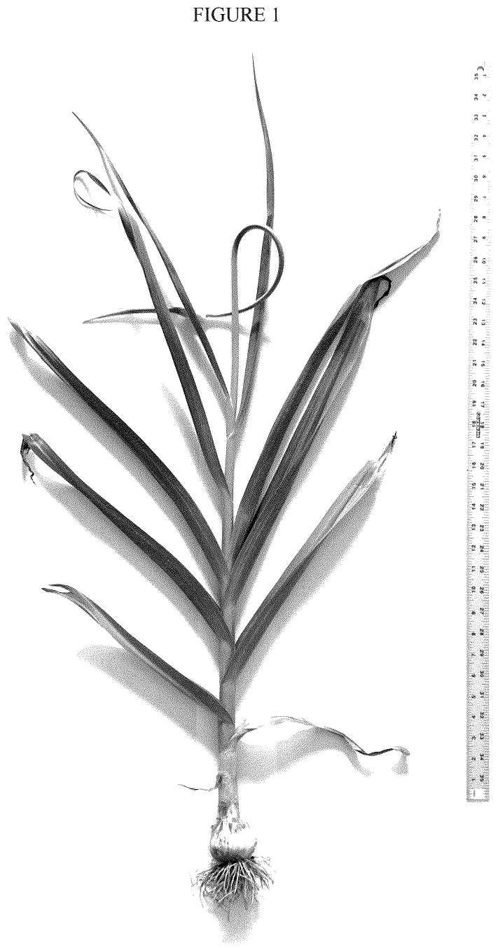

FIG. 2: Slamka Garlic bulb, measuring 6.8 cm in diameter.

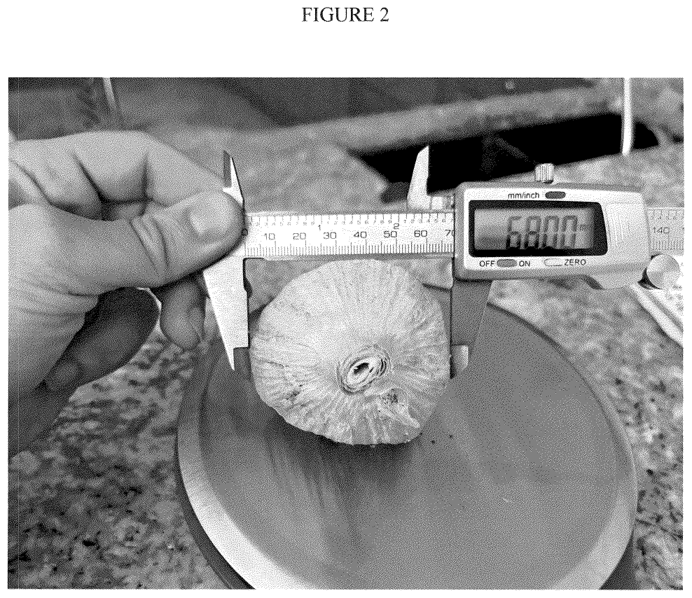

FIG. 3: A larger clove from Slamka Garlic, measuring 4.66 cm in width.

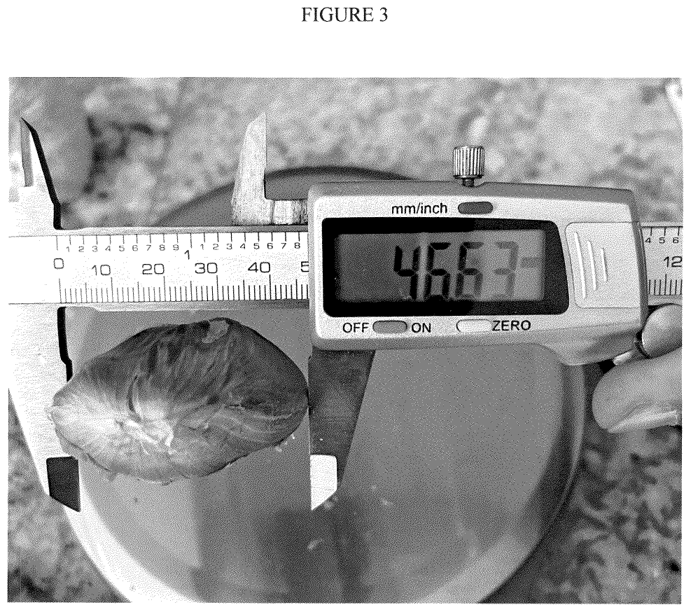

FIG. 4: A partial cluster of cloves after fully cured, demonstrating the clove color and size variation.

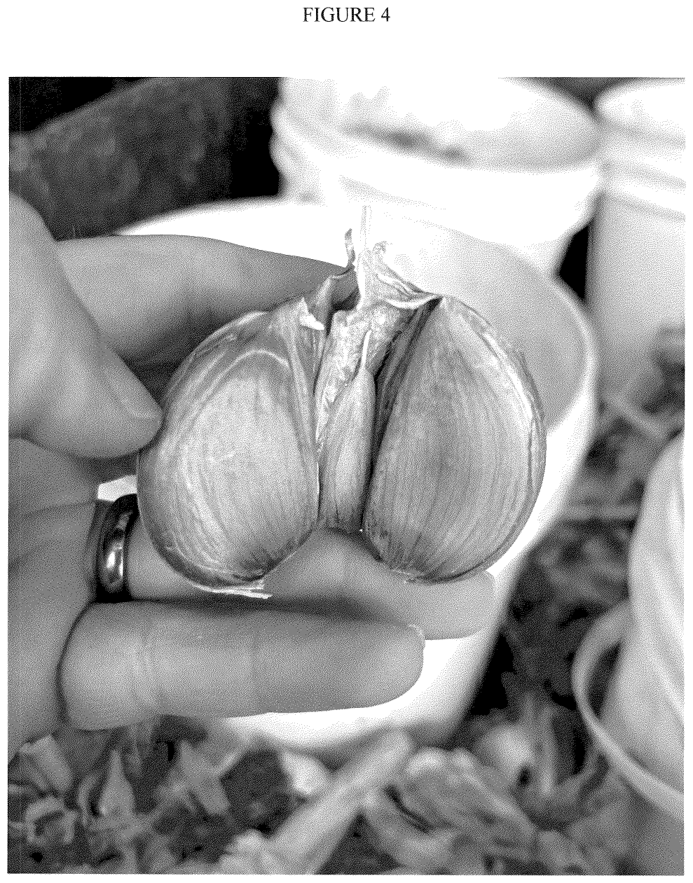

FIG. 5: A horizontal slice of a bulb of Slamka Garlic, 9.5 months after planting, showing the arrangement of 11 individual cloves of varying size.

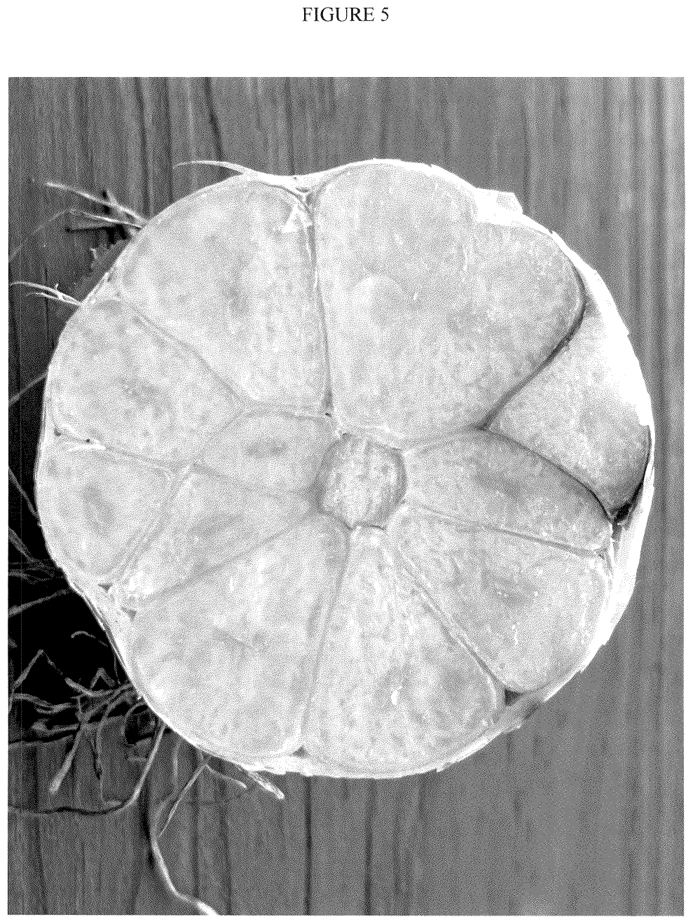

FIG. 6: Slamka Garlic vegetation 4 weeks after planting.

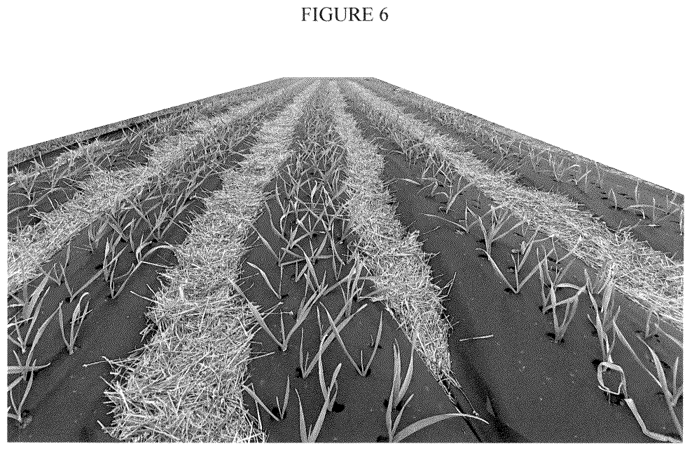

FIG. 7: Umbel with spathe removed, inferior view, showing bulbils with vertical striping. 10 months after planting.

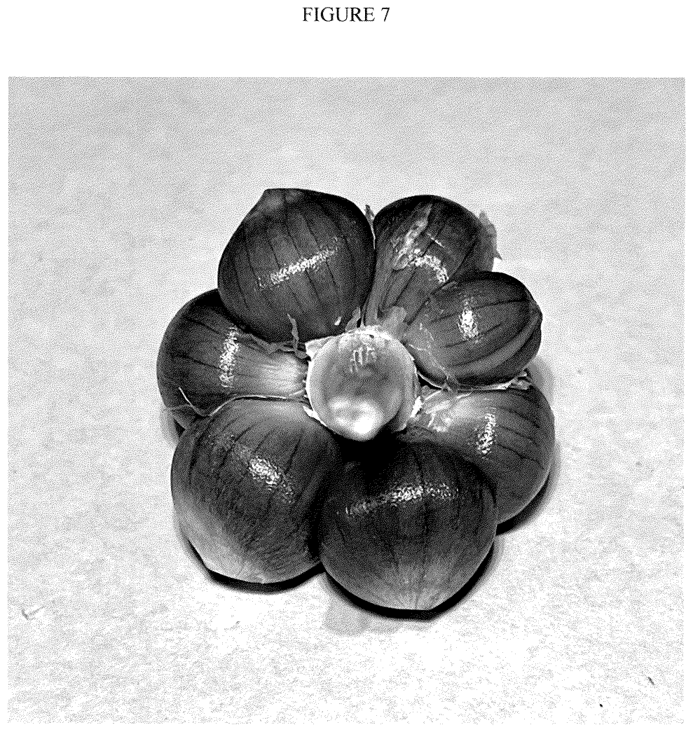

FIG. 8: Umbel with spathe removed, superior view, showing bulbil cluster, flowers, and stems. 10 months after planting.

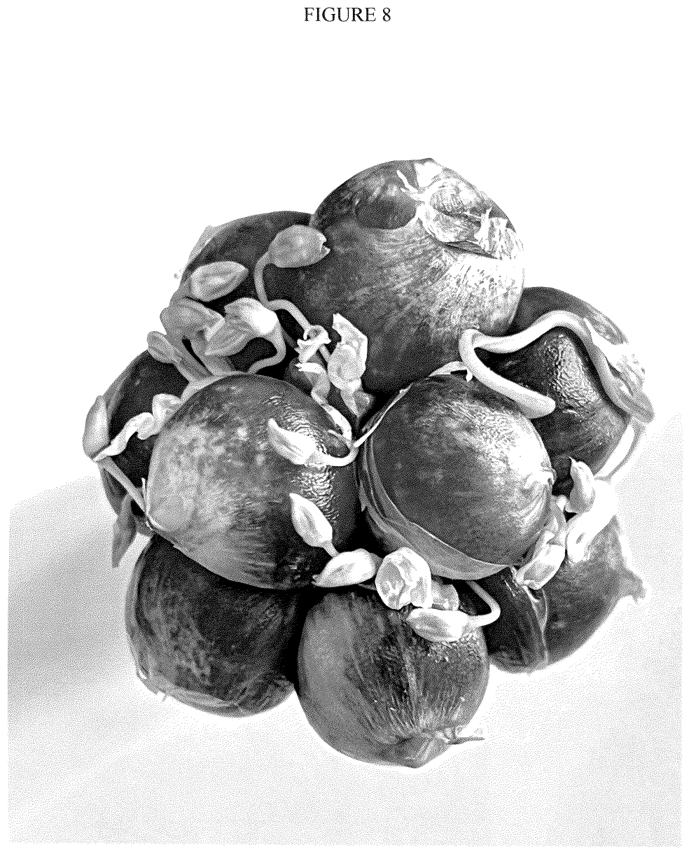

FIG. 9: Flowers on stem with bulbils removed. 10 months after planting.

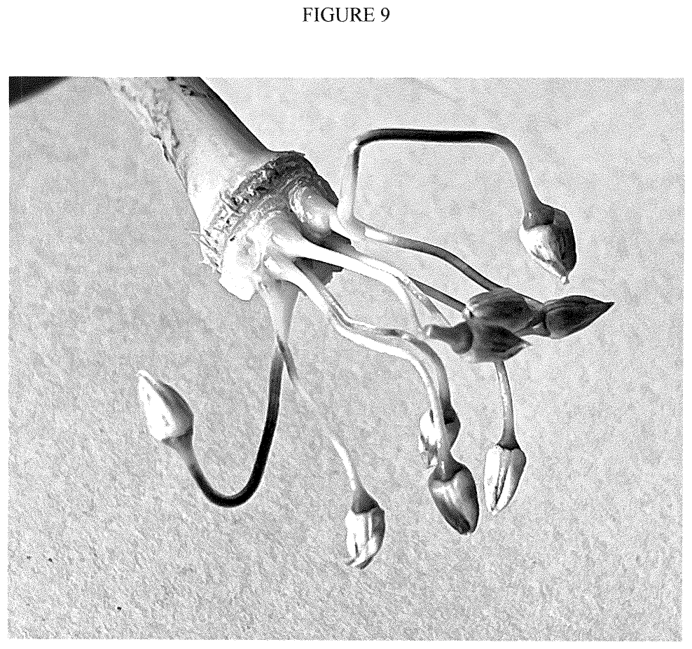

FIG. 10: Flower with petals removed. 10 months after planting.

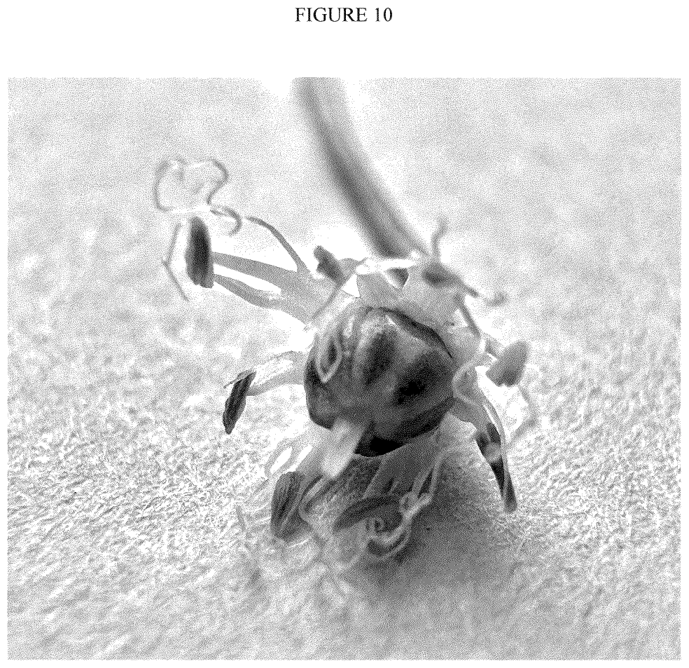

FIG. 11: Flower with petals opened. 10 months after planting.

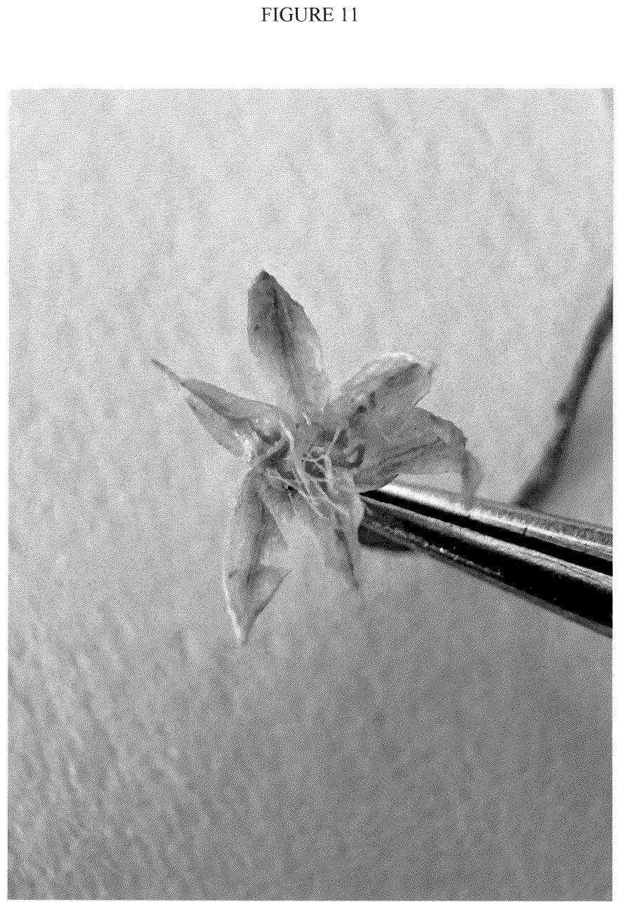

FIG. 12: Flowers on pedicels, 10 months after planting.

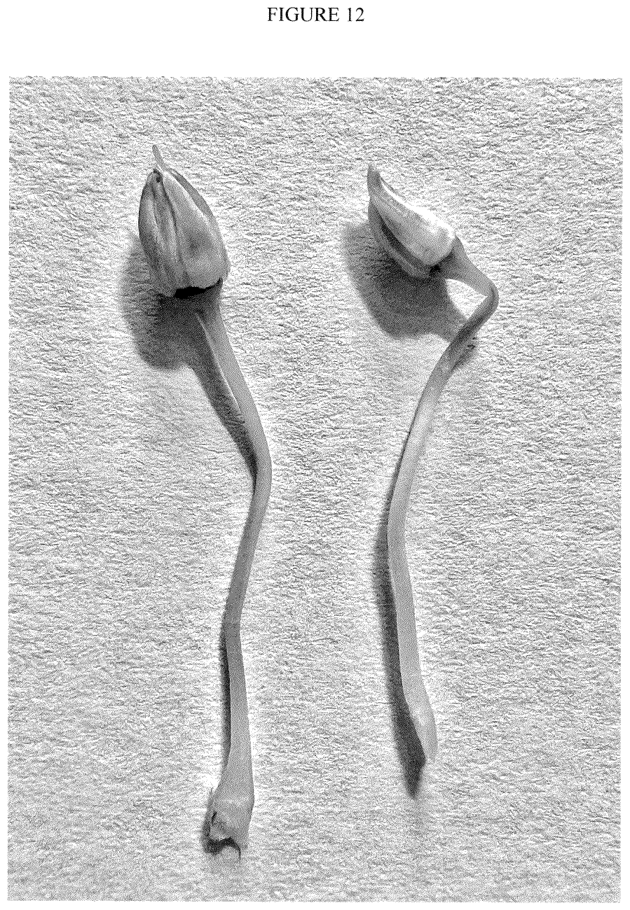

FIG. 13: Garlic Bulb with trimmed roots, showing bulb skin color with purple streaking. 9.5 months after planting.

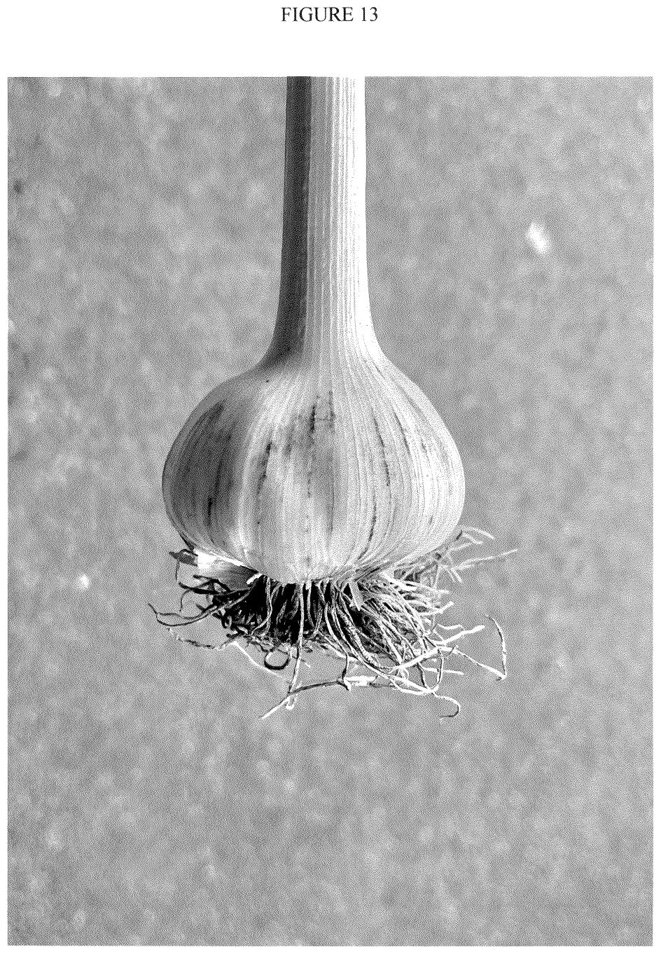

FIG. 14: Garlic bulb after curing, showing dry, off-white outer bulb skin layer with purple hues.

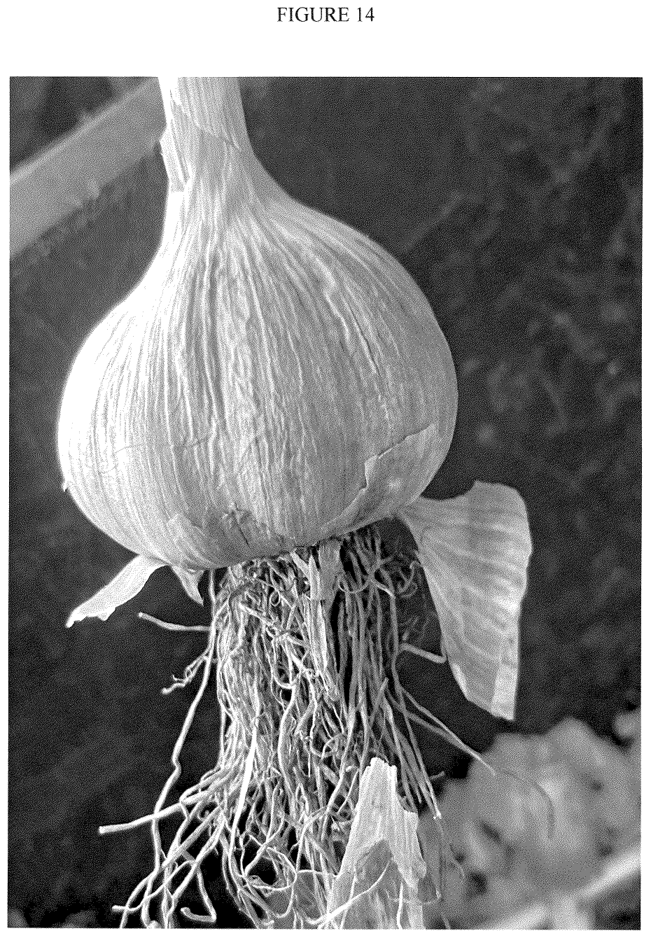

FIG. 15: Harvested scapes showing coloring and curvature.

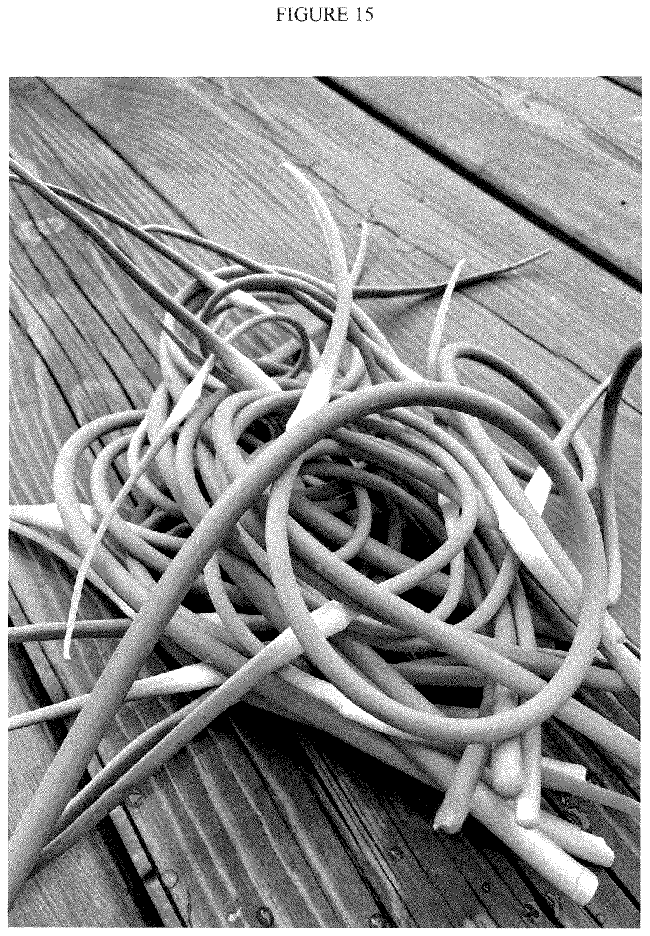

FIG. 16: Scape topset

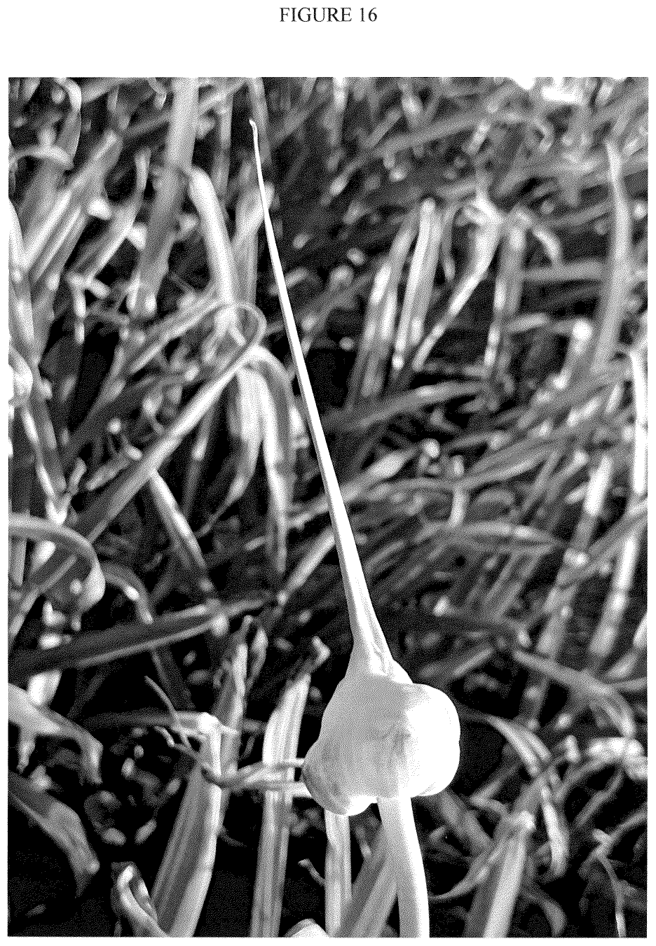

### DETAILED DESCRIPTION OF THE INVENTION

The characteristics of Slamka Garlic may vary depending on environmental factors such as temperature, light intensity, day length, soil composition, or storage conditions, while maintaining consistent genetic traits. It is important to note that Slamka Garlic has not been evaluated under all conceivable environmental conditions. The following data was collected during the 2023-2024 Southeastern Wisconsin growing season.
Botanical description:
   Common name.—Garlic.
   Scientific name.—Allium sativum.
   Family.—Amaryllidaceae.
   Subfamily.—Allioideae.
   Genus.—Allium.
   Species.—A. sativum.
   Variety denomination.—Slamka Garlic.
   Growth habit.—Erect.
   Mature leaf color.—Green with a matte finish.
   Bulb skin color.—Off-white with purple streaks.
   Clove.—Skin: Light to dark brown with hints of purple. The clove exhibits good peeling ability. Flesh: Off-white.
   Scape.—Present. Curvature when young and straightens during maturation. Stem Color: Green. Topset: Oblong and globular umbel with a white spathe. Beak closely matches scape stem color.
   Bulbils.—Large. Skin Color: Varies; brown, deep red/purple, and purple/pink with vertical dark stripes often present. Shape: Ellipsoidal.
   Flowers.—Small, with light pink, purple, and white petals. Anthesis may not occur by the time of harvest.
   Roots.—Develop prior to winter, forming dense, fibrous root structures.
Cultivation details:
   Climate adaptability.—Particularly suited to the temperate climate of Southeastern Wisconsin, withstanding cold winters and hot summers. The performance of Slamka Garlic in other climates has not been tested, and its adaptability outside of Southeastern Wisconsin remains uncertain.
   Soil preference.—Prefers well-drained soil.
   Planting period.—Typically planted in late summer through early fall.
   Harvesting period.—Typically harvested in summer, around July, approximately 10 months after planting.
   Growth.—Sprouts appear approximately 14 days after planting.
   Storage life.—Exhibits a long storage life exceeding 9 months under optimal conditions (cool, dry, and well-ventilated storage).
   Light.—Full sunlight.
   Temperature range.—−11° F. to 99° F.
   Fertilization.—None used.
Plant characteristics:
   Total plant height.—Mean 90.5 cm.
   Bulb characteristics.—Diameter: Can exceed 7.5 cm, with a mean of 7.1 cm. Height: mean 4.7 cm. Weight: Can exceed 150 grams, with a mean of 105 grams.
   Clove characteristics.—Number of Cloves per Bulb: Variable. Typically, 8-14. Clove Size: Variable, can exceed 4.5 cm in width. Flavor Profile: Spicy, aromatic, bold, zesty, and rich.
   Pseudostem characteristics.—Circumference at Base: 6.5 cm-8.5 cm, with a mean of 7.8 cm.
   Leaf characteristics.—Top Leaf Length: mean 44.7 cm. Second Leaf Length: Longer than the top leaf. Bottom Leaf Width: Mean width 4.8 cm. Top Leaf Width: Mean 2.4 cm. Number of Leaves: Typically, 9-11, with an average of 10.
   Root characteristics.—Root Length: Approximate mean 16 cm, can exceed 20 cm.
   Scape characteristics.—Length: Can exceed 75 cm, mean 61.5 cm. Diameter at Base: mean 7.5 mm. Topset Height: mean 17.6 cm. Spathe Opening: About half are open 10 months after planting. Umbel Width: mean 4.4 cm. Umbel Height: mean 2.4 cm. Spathe Beak Length: mean 15.2 cm. Number of Bulbils: Mean 8 bulbils per umbel. Bulbil Weight: Mean 12.4 g/bulbil. Bulbil Height: Mean 15.7 mm. Bulbil Width: Mean 15 mm, bulbil width can exceed 2 cm. Flowers: Average 10 per umbel and 6 petals per flower, mean petal length 4.8 mm. Pedicel: Mean length 18.3 mm, color varies between light purple, green, and white. Stamen: Average 6.5 per flower, mean length 2.2 mm. Anther Length: mean 1.3 mm. Ovary Height: mean 1.7 mm. Ovary Width: mean 1.5 mm. Petal Color: Varies between purple, light purple, and pink.
Productivity:
   Yield.—Approximately 87,693 bulbs per acre with a total weight of 9,208 kg/acre using current growing techniques.

1. A new and distinct variety of Allisen sativum L. plant named ‘Slamka Garlic’, substantially as described and illustrated in the detailed description and figures herein. Progeny or clones of Slamka Garlic that retain the described disease resistance, productivity, and unique physical traits are included in this claim.
